(19) United States
(12) Patent Application Publication
(10) Pub. No.: US 2013/0236546 A1
(43) Pub. Date: Sep. 12, 2013

(54) THYME/PRIMROSE OR IVY FOR TREATING CHRONIC OBSTRUCTIVE PULMONARY DISEASES (COPD)

(71) Applicants: Michael Popp, Lauf-Heuchling, (DE); Elena Lashina, St. Petersburg (RU)

(72) Inventors: Michael Popp, Lauf-Heuchling, (DE); Elena Lashina, St. Petersburg (RU)

(73) Assignee: Bionorica SE, Neumarkt (DE)

(21) Appl. No.: 13/700,351

(22) PCT Filed: May 30, 2011
(86) PCT No.: PCT/EP2011/058852

§ 371 Date: May 29, 2013

(30) Foreign Application Priority Data
May 28, 2010 (DE) 10 2010 021 842.1

(51) Int. Cl. A61K 36/53; A61K 36/25

(52) U.S. Cl. CPC A61K 36/53; A61K 36/25
USPC 424/479; 424/725; 424/474

(57) ABSTRACT

The invention relates to an extract made of thyme (Thymus L.) in combination with primrose (Primula veris) or ivy (Hedera helix) for the prophylaxis and treatment of chronic obstructive pulmonary diseases (COPD) and to a corresponding medicinal product and the use thereof.

The invention relates to an extract made of thyme (Thymus L.) in combination with primrose (Primula veris) or ivy (Hedera helix) for the prophylaxis and treatment of chronic obstructive pulmonary diseases (COPD) and to a corresponding pharmaceutical and to the use thereof.

Bronchipret® (registered trademark of BIONORICA SE) is a known highly potent pharmaceutical—a phytopharmaceutical which contains thyme in combination with primrose or ivy. Bronchipret® is preferably distributed in the form of (film-coated) tablets (dry extract) and syrup or drops (thyme/ivy). The production of the pharmaceutical is known. The secretolytic, bronchospasmolytic, antitussive, antiphlogistic, antibacterial and antiviral effect has been described.

Moreover, R. van den Hoeven (R. van den Hoeven et al, Study of the effect of the lung function of five Austrian saddle horses suffering recurrent airways obstruction (heaves), The Veterinary Record (2003), 152, 555-557) describes the influence of Bronchipret® on the pulmonary function in horses.

Irwin Ziment discusses phytopharmaceuticals in connection with COPD, however provides no indication of the specific suitability of Bronchipret® for the treatment and prophylaxis of COPD (Irwin Ziment, Complementary and alternative medicine therapies for chronic obstructive pulmonary disease Focus Altern Complement Ther 2003; 8: 385-91) It is rather set out that Bronchipret® is especially effective for treating bronchitis. In addition, COPD must be distinguished from bronchitis.

However, a high need exists to provide novel, effective pharmaceuticals for the treatment and prophylaxis of chronic obstructive pulmonary diseases (COPD).

It is therefore the object of the present invention to provide a pharmaceutical that can successfully treat chronic obstructive pulmonary diseases (COPD).

Surprisingly, Bronchipret®, and more particularly a pharmaceutical composition consisting of thyme and ivy, or thyme and primrose, shows a curative and palliative effect for the prophylaxis and treatment of chronic obstructive pulmonary diseases (COPD).

The invention thus relates to a pharmaceutical for the prophylaxis and treatment of chronic obstructive pulmonary diseases (COPD), containing thyme and ivy, or thyme and primrose, and more particularly an extract made of thyme and ivy, or thyme and primrose, as is known for Bronchipret®. The invention further relates to the use of thyme and ivy, or thyme and primrose, or a respective extract thereof, for producing a pharmaceutical for the prophylaxis and treatment of chronic obstructive pulmonary diseases (COPD).

Within the scope of the present invention, chronic obstructive pulmonary diseases (COPD) denotes a group of chronic diseases which are characterized by cough, increased expectoration and shortness of breath under stress conditions, with chronic diseases of the bronchopulmonary system with obstructive ventilatory disorder, wherein inhomogeneous ventilation of the alveoli and increasing pulmonary hyperinflation occurs, all the way to insufficient gas exchange. The disease pattern is characterized in that breathing out (expiration) in particular is labored. The invention is particularly advantageous with acute exacerbations.

According to the invention, asthma, and more particularly bronchial asthma, is therefore likewise included, wherein the attack-like shortness of breath with variable and reversible bronchoconstriction occurs due to inflammation and hyperreactivity of the respiratory tracts, which accompany COPD and can occur, or are even causally related to COPD. The symptoms and clinical characteristics comprise dyspnea, cough with generally thick sputum (contains eosinophil granulocytes, Curschmann's spirals, or Charcot-Leyden crystals).

Also preferred according to the invention are COPD disease forms which are caused by negative environmental influences—which are generally caused by humans or are work-related (mining workers, bakers and the like)—such as dust, dirt particles (exhaust gases), diesel particles (traffic), tar (smoking) and can be the cause of “pneumoconiosis”, “farmer's lung”, “cheese washer's lung” and “smoker's lung” and may be the cause for chronic obstructive pulmonary diseases such as COPD and/or bronchial asthma.

The pharmaceutical according to the invention is thus suited for the prophylaxis and treatment of pneumoconioses, in particular those a) caused by inorganic dust such as coal dust (anthracosis), barite dust (baritosis), iron dust (pulmonary siderosis) or b) by organic dust and accompanied by exogenic allergic alveolitis, such as by mold fungus spores of cheese or in dust of grains, grasses, sugar cane (bagassosis).

The aforementioned plants (plant drugs) according to the invention for the pharmaceutical according to the invention can be obtained, as is customary for the respective plant drug, from preferred parts of the plant, such as the leaf, root and the like, as described in the relevant literature (for example, refer to Ph. Eur., European Pharmacopoeia). In particular aqueous and/or ethanolic extracts can be used, or dry extracts which can be obtained therefrom. For producing the extracts according to the invention, reference is made to the technical teachings, which are the subject matter of EP 1368605B1 and EP 0753306B1.

The pharmaceuticals of the present invention can be produced in an advantageous manner which is known per se, and can be used in form of a pharmaceutical formulation in keeping with the use thereof.

The pharmaceutical formulations of the pharmaceuticals according to the invention are characterized in that oral formulations comprise, for example, sugar-coated tablets, tablets, film-coated tablets, powders, capsules, or liquid dilutions, in particular drops, juices or syrups.

When used for topical applications, in particular sprays, ointments, emulsions, powders, ground substances, liquid or solid preparations for inhalation, compresses, tamponades, tonsil brush solutions or gargling solutions are suited.

The invention further relates to a pharmaceutical formulation containing a pharmaceutical according to the invention. The pharmaceuticals according to the invention can be produced in the form of pharmaceutical preparations in dosage units. This means that the preparation is present in the form of individual parts, such as tablets, sugar-coated tablets, capsules, pills, suppositories and ampoules, the active ingredient content of which corresponds to a fractional or a multiple amount of a single dosage. The dosage units may contain, for example, 1, 2, 3 or 4 single doses or ½, ⅓ or ¼ of a single dose. A single dose preferably contains the amount of active ingredient which is administered with one application and usually corresponds to either the entire daily dose, or one half, one third or one quarter of a daily dose. Non-toxic, inert pharmaceutically suitable carrier substances are to be understood to include solid, semisolid or liquid diluting agents, fillers and formulation adjuvants of any kind.

Tablets, sugar-coated tablets, capsules, pills, granules, suppositories, solutions, juice, suspensions and emulsions, pastes, ointments, gels, creams, lotions, powders and (nasal) sprays shall be mentioned as preferred pharmaceutical formulations. Tablets, sugar-coated tablets, capsules, pills and granules may contain the active ingredient or active ingredients, in addition to the customary carrier substances, such as a) fillers and extenders, for example starches, lactose, sucrose, glucose, mannitol, and silicic acid, b) binders, for example carboxymethyl cellulose, alginates, gelatin, polyvinylpyrrolidone, c) humectants, for example glycerin, d) disintegrating agents, for example agar-agar, calcium carbonate and sodium carbonate, e) dissolution retardants, for example paraffin, and f) resorption accelerating agents, for example quaternary ammonium compounds, g) wetting agents, for example cetyl alcohol, glycerol monostearate, h) adsorption agents, for example kaolin and bentonite, and i) lubricants, for example talcum, calcium stearate and magnesium stearate, and solid polyethylene glycols or mixtures of the substances listed in a) to i).

The tablets, sugar-coated tablets, capsules, pills, and granules can be covered with the customary coatings and encapsulations, optionally containing opaquing agents , and may further be composed in a manner enabling them to release, optionally with delay, the active ingredient or active ingredients only, or preferably, in a defined segment of the intestinal tract, wherein polymer substances and waxes can be used, for example, as embedding compounds.

The active ingredient or the active ingredients can optionally also be present with one or more of the carrier substances mentioned above in microencapsulated form.

In addition to the active ingredient or active ingredients, suppositories may contain the customary water-soluble or water-insoluble carrier substances, for example polyethylene glycols, fats, for example cocoa fat and higher esters (for example, C14-alcohol with C16-fatty acid) or mixtures of these substances.

In addition to the active ingredient or active ingredients, ointments, pastes, creams and gels may contain the customary carrier substances, for example animal and vegetable fats, waxes, paraffins, starch, tragant cellulose derivatives, polyethylene glycols, silicones, bentonites, silicic acid, talcum, and zinc oxide, or mixtures of these substances.

In addition to the active ingredient or active ingredients, powders and sprays may contain the customary carrier substances, for example lactose, talcum, silicic acid, aluminum hydroxide, calcium silicate, and polyamide powder, or mixtures of these substances. Sprays may additionally contain the customary propellants. In addition to the active ingredient or active ingredients, solutions and emulsions may contain the customary carrier substances such as solvents, solubilizers and emulsifiers, for example water, ethyl alcohol, isopropyl alcohol, ethyl carbonate, ethyl acetate, benzyl alcohol, benzyl benzoate, propylene glycol, 1,3-butylene glycol, dimethyl formamide, oils, especially cottonseed oil, peanut oil, corn oil, olive oil, castor oil and sesame oil, glycerin, glycerinformal, tetrahydrofurfuryl alcohol, polyethylene glycols and fatty acid esters of sorbitan, or mixtures of these substances.

In addition to the active ingredient or active ingredients, suspensions may contain customary carrier substances such as liquid diluents, for example water, ethyl alcohol, propylene glycol, suspending agents, for example ethoxylated isostearyl alcohols, polyoxyethylene sorbitol and sorbitan esters, microcrystalline cellulose, aluminum metahydroxide, bentonite, agar-agar and tragant, or mixtures of these substances. The forms of formulations mentioned may also contain colorants, preservatives and odor- and flavor-enhancing additives, for example peppermint oil and eucalyptus oil, and sweeteners, for example saccharin.

Additional advantages and characteristics of the present invention will be apparent from the description of the embodiments. The following examples are provided to describe the invention, without limiting the invention to these examples.

### EXAMPLES

Experimental group: 70 participants (smokers) with work-related COPD (pneumoconiosis), on average 48.9 years old, treatment with Bronchipret® drops, use of the Saint George' Respiratory Questionnaire (SGRG), control group 30 participants;

Dynamics of the Symptoms:

| | | Groups | | | |
|---|---|---|---|---|---|
| | | Experimental group, | | Control group, | |
| | | n = 70 | | n = 3- | |
| | | % of patients, statistics *p < 0.05 | | | |
| | Severity of | | after | | after |
| Symptoms | symptoms | before | therapy | before | therapy |
| cough | 0 | — | 37.07* | — | — |
| | 1 | 12.3 | 10.4* | 15.4 | 16.6 |
| | 2 | 68.76 | 43.25* | 58.5 | 60.4 |
| | 3 | 18.94 | 9.28* | 26.1 | 23 |
| Dyspnea | 0 | 4.28 | 4.28 | — | — |
| | 1 | 2.85 | 51.66* | 13.3 | 30.0. |
| | 2 | 75.72 | 36.6* | 63.4 | 53.3 |
| | 3 | 17.15 | 7.76* | 23.3 | 16.7 |
| Sputum | none | 35.71 | 14.28 | 33.33 | 30.00 |
| | mucoid | 35.71 | 85.72* | 40.0 | 50.0 |
| | mucopurulent | 28.58 | — | 26.67 | 20.00 |

1-8. (canceled)

9. A pharmaceutical which comprises
thyme and ivy, or
thyme and primrose,
wherein the pharmaceutical is useful for the prophylaxis and treatment of chronic obstructive pulmonary diseases and/or pneumoconioses and/or asthma in humans.

10. A pharmaceutical which comprises
extracts made of thyme and ivy, or
extracts made of thyme and primrose,
wherein the pharmaceutical is useful for the prophylaxis and treatment of chronic obstructive pulmonary diseases and/or pneumoconioses and/or asthma in humans.

11. The pharmaceutical according to claim 9 for the prophylaxis and treatment of pneumoconiosis, farmer's lung, cheese washer's lung and smoker's lung.

12. The pharmaceutical according to claim 9 for the prophylaxis and treatment of COPD.

13. The pharmaceutical according to claim 9 for the prophylaxis and treatment of acute exacerbations, and bronchial asthma.

14. A process for the prophylaxis and treatment of chronic obstructive pulmonary diseases and/or pneumoconioses in humans which comprises administering the pharmaceutical as claimed in claim 9 to humans.

15. The pharmaceutical according to claim 9 which further comprises additional additives or adjuvants.

16. A process for the prophylaxis and treatment of chronic obstructive pulmonary diseases and/or pneumoconioses in humans which comprises administering the pharmaceutical as claimed in claim 10 to humans.

17. A pharmaceutical according to claim 9, wherein the pharmaceutical is present in a pharmaceutical formulation.

18. A pharmaceutical according to claim 9, wherein the pharmaceutical is present in an oral formulation such as sugar-coated tablets, tablets, film-coated tablets, capsules, liquid dilutions, or a formulation for topical application, liquid or solid preparations for inhalation, compresses, tamponades, tonsil brush solutions, gargling solutions or rinsing solutions.

19. A pharmaceutical according to claim 9, wherein the pharmaceutical a sugar-coated tablet, film-coated tablet, capsule, drops, juices, syrups, sprays, ointments, emulsions, powder, ground material, liquid or solid preparations for inhalation, compresses, tamponades, tonsil brush solutions, gargling solutions or rinsing solutions.

20. A pharmaceutical composition, containing the pharmaceutical according to claim 9.
